(19) United States
(12) Patent Application Publication
(10) Pub. No.: US 2015/0366901 A1
(43) Pub. Date: Dec. 24, 2015

(54) Methods of Treating Heart Failure

(71) Applicant: The Trustees of the University of Pennsylvania, Phildelphia, PA (US)

(72) Inventor: Julio A. CHIRINOS, Gamet Valley, PA (US)

(21) Appl. No.: 14/765,583

(22) PCT Filed: Feb. 7, 2014
(86) PCT No.: PCT/US2014/015300

§ 371 Date: Aug. 4, 2015

Related U.S. Application Data
(60) Provisional application No. 61/761,863, filed on Feb. 7, 2013.

(51) Int. Cl. A61K 33/00; A61K 45/06

(52) U.S. Cl. CPC A61K 33/00; A61K 45/06

(57) ABSTRACT

The present invention relates to the discovery that the administration of nitrate is an effective therapy for improving short-term and long-term outcomes in patients with heart failure, including heart failure with preserved ejection fraction (HFpEF). Thus, the present invention provides compositions and methods for the treatment or prevention of heart failure, including HFpEF, in a subject in need thereof.

### CROSS-REFERENCE TO RELATED APPLICATIONS

This application claims priority to U.S. Provisional Application Ser. No. 61/761,863, filed Feb. 7, 2013, which is incorporated by reference herein in its entirety.

### BACKGROUND OF THE INVENTION

Heart failure (HF) with preserved ejection fraction (HFpEF) is a major epidemic. HF affects ˜2% of the western population and 10% of adults aged >75 years (Lam et al., 2011, Eur. J. Heart Failure 13:18-28). HF is the most common cause of hospitalization in adults >65 years of age (Lam et al., 2011, Eur. J. Heart Failure 13:18-28). Approximately 54% of patients with HF (Lam et al., 2011, Eur. J. Heart Failure 13:18-28; Vasan et al., 1995, J. Am. College Cardiol. 26:1565-1547; Redfield et al., 2003, Jama 289:194-202; Kitzman et al., 2001, Am. J. Cardiol. 87:413-419; Devereux et al., 2000, Am. J. Cardiol. 86:1090-1096; Ceia et al., 2002, Eur. J. Herat Failure 4:531-539; Mosterd et al., 1999, Eur. Heart J. 20:447-455; Morgan et al., 1999, BMJ 318:368-372; Cortina et al., 2001, Am. J. Cardiol. 87:1417-1419; Kupari et al., 1997, J. Intern. Med. 241:387-394) and 46-51% of patients hospitalized for acute HF have HFpEF (Lam et al., 2011, Eur. J. Heart Failure 13:18-28; Fonarow et al., 2007, J. Am. College Cardiol. 50:768-777; Yancy et al., 2006, J. Am. College Cardiol. 47:76-84; Lenzen et al., 2004, Eur. Heart J. 25:1214-1220). The prevalence of HFpEF in the general population is as high as 1.1-5.5% (Lam et al., 2011, Eur. J. Heart Failure 13:18-28; Owan et al., 2005, Prog. Cardiofasc. Dis. 47:320-332).

The prevalence of HFpEF will continue to increase. The number of new HF cases in the US has increased from 348,000 in 2000 to 670,000 in 2007 (Lam et al., 2011, Eur. J. Heart Failure 13:18-28; Lloyd-Jones et al., 2010, Circulation 121:586-613) (93% increase), greatly exceeding previous forecasts and suggesting that a further dramatic increase should be expected in the next few decades (Lam et al., 2011, Eur. J. Heart Failure 13:18-28; Lloyd-Jones et al., 2010, Circulation 121:586-613). Assuming that half the caseload of HF consists of HFpEF, an equal increase in HFpEF burden can be projected. Even these may be conservative estimates, since the relative prevalence of HFpEF (as a proportion of the total burden of HF cases) is increasing as the population ages (Lam et al., 2011, Eur. J. Heart Failure 13:18-28; Owan et al., 2005, Prog. Cardiofasc. Dis. 47:320-332). A study from Olmsted County, Minn., indicated that HFpEF comprised 38% of all HF cases in 1987, increasing to 54% in 2001 (Lam et al., 2011, Eur. J. Heart Failure 13:18-28; Owan et al., 2005, Prog. Cardiofasc. Dis. 47:320-332). In the same time frame, survival was noted to improve in patients with HF with reduced ejection fraction, but not in those with HFpEF. Therefore, although already an epidemic, a further dramatic increase in the prevalence of HFpEF is anticipated (Lam et al., 2011, Eur. J. Heart Failure 13:18-28).

HFpEF is a malignant disease with high mortality. Studies have consistently demonstrated high annual mortality rates in patients with HFpEF, ranging from ˜3.5-6% in large randomized trials (Cleland et al., 2006, Eur. Heart J. 27:2338-2345; Massie et al., 2008, N. Eng. J. Med. 359:2456-2467; Yusuf et al., 2003, Lancet 262:777-781) to ˜15% in the Framingham Heart Study (Lee et al., 2009, Circulation 119:3070-3077). A meta-analysis of 7,688 HFpEF patients followed for ˜4 years reported an annual mortality rate of ˜8% (Lam et al., 2011, Eur. J. Heart Failure 13:18-28). During decompensations, 90-day mortality and re-hospitalization rates are 5-9.5% (Fonarow et al., 2007, J. Am. College Cardiol. 50:768-777; Perez de Isla et al., 2008, J. Cardiovasc. Med 9:1011-1015; Tsuchihashi-Makaya et al., 2009, Circ. J. 73:1893-1900; Bhatia et al., 2005, N. Engl. J. Med. 354:259-269) and ˜29% (Fonarow et al., 2007, J. Am. College Cardiol. 50:768-777, Lenzen et al., 2004, Eur. Heart J. 25:1214-1220) respectively. Cardiovascular causes account from ˜50% deaths in HFpEF (Lam et al., 2011, Eur. J. Heart Failure 13:18-28). Multiple therapies that provide substantial clinical benefit in HF with reduced ejection fraction are available. In contrast, there are currently no effective dietary or pharmacologic interventions that improve long-term outcomes in patients with HFpEF (Oghlakian et al., 2011, Mayo Clin Proc. 86:531-539).

Exercise intolerance is the hallmark of HFpEF and determines a poor quality of life (Hoekstra et al., 2011, European J. Heart Fail. 13:1013-1018; Lewis et al., 2007, European J. Heart Fail. 9:83-91; Kitzman et al., 2002, JAMA 288:2144-2150; Phan et al., 2012, Int. J. Cardiol. 158:337-343) Therefore, enhancing exercise capacity in this population is a key objective with immediate clinical relevance. This goal requires consideration of the pathophysiology of exercise intolerance in HFpEF. The early pathophysiologic paradigm was that increases in left ventricular (LV) filling pressure during exercise were not accompanied by increases in end-diastolic volume, leading to a failure to recruit the Frank-Starling mechanism and to augment stroke volume (Kitzman et al., 1991, J. Am. College Cardiol. 17:1065-1072). Various subsequent studies, however, failed to show abnormalities in exercise end-diastolic LV volume (Borlaug et al., 2006, Circulation 114:2138-2147; Ennezat et al., 2008, J. Card. Fail. 14:475-480; Maeder et al., 2010, J. Am. Coll. Cardiol. 56:855-863) and stroke volume reserve in HFpEF (Borlaug et al., 2006, Circulation 114:2138-2147 Maeder et al., 2010, J. Am. Coll. Cardiol. 56:855-863; Bhella et al., 2011, Eur. J. Heart Fail. 13:1296-1304). Subsequent studies reported the presence of chronotropic incompetence, leading to an abnormal cardiac output reserve (Borlaug et al., 2006, Circulation 114:2138-2147; Maeder et al., 2010, J. Am. Coll. Cardiol. 56:855-863; Bhella et al., 2011, Eur. J. Heart Fail. 13:1296-1304). However, available data are somewhat conflicting, since neither exercise, chronotropic incompetence (Ennezat et al., 2008, J. Card. Fail. 14:475-480) nor cardiac output reserve have been consistent findings (Bhella et al., 2011, Eur. J. Heart Fail. 13:1296-1304). Thus, rather than resulting exclusively from cardiac abnormalities, HFpEF is now seen a complex multi-organ disease and there is a great need to understand and target peripheral abnormalities in this condition.

Exercise arterial vasodilator (“afterload”) reserve is abnormal in HFpEF. During exercise, LV afterload (arterial resistance and impedance) must decrease to accommodate increases in flow without excessive increments in pressure. In several studies, compared to normal controls (Maeder et al., 2010, J. Am. Coll. Cardiol. 56:855-863; Borlaug et al., 2010, J. Am. Coll. Cardiol. 56:845-854, author reply 156-158), age-matched hypertensive subjects without HF (Ennezat et al., 2008, J. Card. Fail. 14:475-480; Borlaug et al., 2010, J. Am. Coll. Cardiol. 56:845-854, author reply 156-158), or age- and comorbidity-matched controls (Borlaug et al., 2006, Circulation 114:2138-2147), patients with HFpEF demonstrated blunted exercise-induced decreases in systemic vascular resistance, indicating impaired vasodilatory responses during exercise.

Skeletal muscle flow and oxygen delivery and extraction are important components of the normal exercise response (Poole et al., 2012, Am. J. Physiol. Hert Circ. Physiol. 302:H1050-1063), and depends on the vasodilatory response in locomotive muscle, allowing it to effectively “compete” for the available cardiac output (Poole et al., 2012, Am. J. Physiol. Hert Circ. Physiol. 302:H1050-1063). This process requires working skeletal muscle vasculature to overcome humoral and reflex-mediated vasoconstriction (Poole et al., 2012, Am. J. Physiol. Hert Circ. Physiol. 302:H1050-1063). NO bioavailability and release is a key mechanism mediating this response (Poole et al., 2012, Am. J. Physiol. Hert Circ. Physiol. 302:H1050-1063). Importantly, impaired vascular responses within muscle can have dramatic consequences for O₂ extraction, creating a marked imbalance between O₂ delivery and requirement in muscle and resulting in a large O₂ deficit, accentuated intracellular metabolic perturbations and enhanced glycogenolysis even at low levels of activity (Poole et al., 2012, Am. J. Physiol. Hert Circ. Physiol. 302:H1050-1063).

Although blood flow to relevant muscle groups is clearly important during exercise, blood flow (Q) to active muscles is not homogeneous, being greater in highly oxidative muscles, which normally demonstrate greater endothelium-dependent vasodilatation (Muller-Delp, 2006, Microcirculation 13:301-314; Poole et al., 2007, Exp. Physiol. 92:341-346). Dysregulation of these control processes provides an excess flow and therefore O₂ delivery to less metabolically active muscles with diminished ability for O₂ exchange, thus reducing muscle and whole-body fractional O₂ extraction (Poole et al., 2007, Exp. Physiol. 92:341-346).

O₂ extraction appears to be a key abnormality in the pathophysiology of exercise intolerance in HFpEF. Peak O₂ uptake (VO₂), the most widely accepted index of aerobic capacity, is reduced in HFpEF (Kitzman et al., 1991, J. Am. College Cardiol. 17:1065-1072; Borlaug et al., 2006, Circulation 114:2138-2147; Maeder et al., 2010, J. Am. Coll. Cardiol. 56:855-863; Bhella et al., 2011, Eur. J. Heart Fail. 13:1296-1304 Borlaug et al., 2010, J. Am. Coll. Cardiol. 56:845-854). As the product of cardiac output and arterial-venous O₂ content difference, a depressed peak VO₂ may reflect a defect in O₂ tissue delivery or extraction (predominantly in skeletal muscle), a limitation in cardiac output during exercise, or both. Recently, studies from 3 separate laboratories, using 3 different techniques, showed that patients with HFpEF demonstrate a reduced peak exercise arterio-venous O₂ gradient (Kitzman et al., 1991, J. Am. College Cardiol. 17:1065-1072; Bhella et al., 2011, Eur. J. Heart Fail. 13:1296-1304; Haykowsky et al., 2011, JAC 58:265-274). This indicates that for any given cardiac output during exercise, HFpEF patients have a lower O₂ peripheral oxygen extraction. Furthermore, in a randomized, controlled trial of exercise training in elderly patients with HFpEF (Haykowsky et al., 2012, J. Am. Coll. Cardiol. 60:120-128), the improvement in peak exercise capacity associated with endurance exercise was related primarily to an increased peak arterio-venous O₂ gradient, and not to an enhanced cardiac output, indicating that peripheral vascular and/or skeletal muscle function were improved, resulting in enhanced O₂ transport and/or O₂ utilization by the active skeletal muscle. A pharmacologic intervention to enhance O₂ transport and/or its efficient utilization by skeletal muscle, has not yet been proposed/identified or tested in HFPEF.

The arterial tree is well known to directly determine pulsatile LV afterload (Nichols et al., 2005, McDonald's blood flow in arteries. Theoretical, experimental and clinical principles, Oxford University Press; Kass, 2005, Hypertension 46:185-193; Mitchell, 2009, Med. Biol. Eng. Comput, 47:153-163; Chirinos and Segers, 2010, Hypertension 56:563-570; Chirinos and Segers, 2010, Hypertension 56:555-562; Mitchell, 2004, Curr. Hypertens. Rep. 6:436-441; Mitchell, Med. Biol. Eng. Comput. 47:153-163; Nichols and Vlachopolous, 2011, McDonald's blood flow in arteries. Theoretical, experimental and clinical principles, Hodder Arnold; Westerhof et al., 2009, Med. Biol. Eng. Comput. 47:131-141; Segers et al., 2000, Hyptertension 36:760-765; Mitchell, 2004, Curr. Hyptertens. Rep. 6:436-441; Chirinos, 2012, J. Cardiovasc. Transl. res. 5:243-255; Segers et al., Proc. Inst. Mech. Eng. H. 222:417-428) increase the systolic LV myocardial wall stress (Chirinos et al., 2012, Hyptertension 60:64-70) and κ₂ consumption, affecting the matching between the ventricle and arterial system, which influences myocardial O₂ supply/demand and cardiac efficiency (Kelly et al., 1992, Circ. Res. 71:490-502). Several abnormalities in the arterial tree that promote an increased cardiac workload have been identified. Subjects with exertional dyspnea or those with frank HFpEF demonstrate increased large artery stiffness (Weber et al., 2008, Am. J. Hypertens. 21:1194-1202; Hundley et al., 2001, J. Am. College Cardiol. 38:796-802). Furthermore, increased large artery stiffness is closely associated with diminished peak exercise O₂ consumption (R=0.79) (Hundley et al., 2001, J. Am. College Cardiol. 38:796-802).

The pulse wave generated by the LV travels forward in arteries and is partially reflected at sites of impedance mismatch (i.e., bifurcations, points of change in arterial size or wall stiffness). Wave reflections arise predominantly in middle-sized conduit arteries and travel back to the heart, merging into a discrete reflected wave (Chirinos and Segers, 2010, Hypertension 56:563-570; Chirinos and Segers, 2010, Hypertension 56:555-562; Nichols and Vlachopolous, 2011, McDonald's blood flow in arteries. Theoretical, experimental and clinical principles. Hodder Arnold). The reflected wave affects LV afterload and alters the loading sequence due to the wave transit time from the heart to reflection sites and back to the proximal aorta, wave reflections arrive back at heart while the LV is still ejecting blood in mid-to-late systole (Nichols and Vlachopolous, 2011, McDonald's blood flow in arteries. Theoretical, experimental and clinical principles. Hodder Arnold; Chirinos and Segers, 2010, Hypertension 56:563-570). Wave reflections thus increase the mid-to-late systolic workload of the LV and profoundly impact the LV loading sequence (late relative to early systolic load).

Late systolic load from wave reflections leads to LV hypertrophy. For any given level of systolic pressure, late-systolic load exerts deleterious effects on the LV (Nichols and Vlachopolous, 2011, McDonald's blood flow in arteries. Theoretical, experimental and clinical principles. Hodder Arnold; Kobayashi et al., 1996, Circulation 94:3362-3368; Gillebert and Lew, 1991, Am. J. Physiol. 261:H805-813). In a Wistar rat model, constriction of the abdominal aorta (which caused prominent late systolic loading from a reflected wave at the distal constriction site) resulted in much greater LV hypertrophy and fibrosis than constriction of the aortic arch (which increased the early systolic load) despite identical peak LV pressures. These causal findings are strongly supported by human data (Hashimoto et al., 2008, J. Hypertens. 26:1017-1024) indicating that changes in wave reflection magnitude during antihypertensive therapy strongly predict regression of LV mass, independently of blood pressure reduction. Of note, standard antihypertensive medications have highly inconsistent effects on wave reflections.

It has been shown that late systolic inflation of an aortic balloon impairs tau (gold standard measure of LV relaxation) much more than early systolic inflation in dogs, demonstrating a cause-effect relationship between late systolic load and diastolic dysfunction (Gillebert and Lew, 1991, Am. J. Physiol. 261:H805-813). In support of these causal findings, wave reflections are independently associated with diastolic dysfunction in human clinical cohorts (Weber et al., 2008, Am. J. Hypertens. 21:1194-1202, Fukuta et al., 2010, Circ. J. 74:1900-1905).

NO formation occurs via two pathways in mammals: (1) NO synthases (NOS) catalyze the formation of NO from L-arginine and O₂ (Chirinos, 2012, J. Cardiovasc. Transl. Res. 5:243-255; Chirinos et al., 2012, Hypertension 60:64-70; Ordonez et al., 2011, Anticancer Res. 31:3607-3613; Lundberg et al., 2008, Nat. Rev. Drug Discov. 7:156-167); and (2) circulating nitrate (previously considered an inert product of NO metabolism) (Ordonez et al., 2011, Anticancer Res. 31:3607-3613) can be converted to NO through the nitrate-nitrite-NO pathway, which is largely independent of NOS (Lundberg et al., 2008, Nat. Rev. Drug Discov. 7:156-167; Cosby et al, 2003, Nat. Med. 9:1498-1505; Machha and Schechter, 2012, Nutr. Rev. 70:367-372; Tang et al., 2011, Curr. Opin. Lipidol. 22:11-15; Weitzberg et al., 2010, Anesthesiology 113:1460-1475; Lundberg et al., 2011, Cardiovasc. Res. 89:525-532). Ingested inorganic nitrate is readily absorbed across the upper gastrointestinal tract. Furthermore, oral cavity commensal bacteria reduce nitrate to nitrite, which has a high oral bioavailability (>95%) (Lundberg et al., 2011, Cardiovasc. Res. 89:525-532; Dibble et al., 2011, Chest 140:310-316; Rubin et al., 2011, Am. J. Kidney Dis. 57:488-497; Durand et al., 2010, Contraception 82:526-533). Nitrite present in the blood stream is reduced directly to NO, a reaction catalyzed by several molecules, including deoxygenated myoglobin (Totzeck et al., 2012, Circulation 126:325-334; Shiva et al., 2007, Circ. Res. 100:654-661; Rassaf et al., 2007, Circ. Res. 100:1749-1754; Hendgen-Cotta et al., 2008, Proc. Natl. Acad. Sci. USA 105:10256-10261), deoxygenated hemoglobin (Gladwin and Kim-Shapiro, 2008, Blood 112:2636-2647), xanthine oxidoreductase (Webb et al., 2004, Proc. Natl. Acad. Sci. USA 101:13683-13688), respiratory chain enzymes of mitochondria (Kozlov et al., 1999, FEBS Lett. 454:127-130), aldehyde oxidase (Zweier et al., 2010, Nitric Oxide 22:83-90), carbonic anhydrase (Aamand et al., 2009, Am. J. Physiol. Heart Circ. Physiol. 297:H2068-2074), vitamin C (Carlsson et al., 2001, Nitric Oxide 5:580-586), polyphenols (Gago et al., 2007, Free Radic. Biol. Med. 43:1233-1242; Gago et al., 2008, Free Radic. Biol. Med. 45:404-412) and even endothelial NO synthase (Gautier et al., 2006, Biochem. Biophys. Res. Commun. 341:816-821; Vanin et al., 2007, Cell Mol. Life Sci. 64:96-103). Nitrate circulates in plasma and has a half-life of ˜5 h. Up to 25% of all circulating nitrate is actively taken up by the salivary glands and concentrated in the saliva (entering an entero-salivary cycle) (Betalleluz-Pallardel et al., 2012, Food Sci. Technol. Int. 18:271-280), while the rest is excreted by the kidneys.

Vegetables are the dominant source of nitrate in the diet (>80%). Leafy green vegetables and beetroot, in particular, contain high amounts of nitrates (Lundberg et al., 2008, Nat. Rev. Drug Discov. 7:156-167; Lundberg et al., 2011, Cardiovasc. Res. 89:525-532). Increased dietary intake of nitrate can increase systemic nitrate and nitrite levels dramatically and “fuel” the nitrate-nitrite pathway, even after a single nitrate-rich beverage (Lundberg et al., 2011, Cardiovasc. Res. 89:525-532; Dibble et al., 2011, Chest 140:310-316; Rubin et al., 2011, Am. J. Kidney Dis. 57:488-497; Durand et al., 2010, Contraception 82:526-533). Although NOS-derived NO is rapidly oxidized to form nitrite (NO₂⁻) and nitrate (NO₃⁻) (Chirinos, 2012, J. Cardiovasc. Transl. Res. 5:243-255; Ordonez et al., 2008, Nat. Rev. Drug Discov. 7:156-167), it makes a limited contribution to the circulating nitrate pool.

The conversion of nitrite to NO is enhanced under hypoxic conditions. Exercising muscle is featured by a low pO₂ (Lundberg et al., 2011, Cardiovasc. Res. 89:525-532), which favors the formation of NO from circulating nitrite. Xanthine oxidoreductase converts nitrite to NO when O₂ levels are low (Webb et al., 2004, Proc. Natl. Acad. Sci. USA 101:13683-13688). Similarly, deoxyhemoglobin supports the reduction of nitrite to NO and is thus thought to play a key role in modulating small resistance vessels (particularly of muscular vascular beds), where O₂ extraction from the circulation to the tissues is most marked. Here, the O₂ saturation of hemoglobin approaches the P50 (the O₂ concentration at which half the hem is saturated), an optimum balance point between the greater reductive potential of hem in the R (oxy) state tetramer and the number of un-ligated deoxy-hem sites necessary for nitrite binding (which are more plentiful in the T-state tetramer). This results in near-maximal conversion rates of nitrite to NO and hence vasodilatation. Similarly, NO by deoxy-myoglobin enhances blood flow to skeletal muscle and matches O₂ supply to increased metabolic demands under hypoxic conditions (Totzeck et al., 2012, Circulation 126:325-334). Interestingly, NO production via the classic arginine pathway is blocked in inhibited by hypoxia, whereas endothelial NOS-mediated NO production from nitrite reduction is enhanced in the absence of hypoxia (Vanin et al., 2007, Cell Mol. Life Sci. 64:96-103). Thus, endogenous nitrite is a physiological effector of hypoxic vasodilation via NO release, which is independent of the L-arginine pathway and largely independent of NOS. (Cosby et al., 2003, Nat. Med. 9:1498-1505).

Nitrates enhance the efficiency of mitochondria and reduce the O₂ cost of exercise in normal subjects (Bailey et al., 2009, J. Appl. Physiol. 107:1144-1155). Therefore, not only does nitrite-mediated vasodilation seem ideal to enhance O₂ delivery to exercising muscle, but multiple studies demonstrate that nitrates reduce the O₂ cost of low- and high-intensity exercise, including submaximal cycling (Bailey et al., 2009, J. Appl. Physiol. 107:1144-1155; Larsen et al., 2007, Acta Physiol. (Oxf.) 191:59-66; Vanhatalo et al., 2010, Am. J. Physiol. Regul. Integr. Comp. Physiol. 299:R1121-1131), knee extensor exercise (Bailey et al., 2010, J. Appl. Physiol. 109:135-148), walking and running (Lansley et al., 2011, Cell Metab. 13:149-159) in healthy volunteers. This effect is not due to increased anaerobic metabolism (Larsen et al., 2007, Acta Physiol. (Oxf.) 191:59-66), indicating an intrinsic improvement in energetic efficiency. Recently, nitrates were shown to reduce the ATP cost of muscle force production (Bailey et al., 2010, J. Appl. Physiol. 109:135-148) and to enhance the efficiency of skeletal muscle mitochondria in humans (Larsen et al., 2011, Cell Metab. 13:149-159).

Inorganic nitrites/nitrates improve exercise capacity in normal subjects. Dietary supplementation either with sodium nitrate or nitrate-rich beetroot juice has been shown to extend time-to-exhaustion during high-intensity constant-work-rate exercise by about 15%-25% (Bailey et al., 2009, J. Appl. Physiol. 107:1144-1155; Bailey et al., 2010, J. Appl. Physiol. 109:135-148; Lansley et al., 2011, Cell Metab. 13:149-159) and more recently, to enhance athletic performance (fastest possible time for healthy subjects to complete a given distance in a bicycle ergometer) (Lansley et al., 2011, Med. Sci. Sports Exerc. 43:1125-1131). Dietary nitrites also increased peak power output during incremental exercise after chronic supplementation, indicating the potential for a sustained benefit (Vanhatalo et al., 2010, Am. J. Physiol. Regul. Integr. Comp. Physiol. 299:R1121-1131). Whereas these exercise-enhancing effects in healthy subjects are well documented, this approach has never been tested to enhance exercise tolerance in HFpEF.

In addition to their exercise-enhancing mechanistic effects, dietary nitrates exert peripheral arterial effects with a potential for chronic “disease-modifying” benefits in HFpEF. A recent placebo-controlled randomized study among healthy volunteers demonstrated that ingestion of 8 mmol of inorganic nitrate increased plasma nitrates 3 hours post-ingestion and this was associated with a decrease in aortic pulse wave velocity (gold standard index of aortic stiffness) (Bahra et al., 2012, Nitric Oxide 26:197-202). This human study is in line with another recent study in which sodium nitrite supplementation for 3 weeks reduced aortic pulse wave velocity (PWV) in old mice (478-384 AU) to values closer to their young counterparts (332 AU) (Sindler et al., 2011, Aging Cell 10:429-437).

Thus, there remains a need in the art for improved compositions and methods of treating HFpEF. The present invention addresses these unmet needs in the art.

### SUMMARY OF THE INVENTION

The invention relates to the discovery that inorganic nitrate or nitrite is an effective therapy for improving exercise tolerance, symptoms, quality of life, and/or long-term outcomes in patients with heart failure (HF), including HF with preserved ejection fraction (HFpEF). Thus, in one embodiment, the invention is a method of treating or preventing heart failure in a subject in need thereof, the method comprising administering to the subject a therapeutically effective amount of a composition comprising at least one selected from the group consisting of inorganic nitrate and inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In another embodiment, the invention is a method of improving exercise tolerance in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In one embodiment, the invention is a method of reducing large artery stiffness in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In another embodiment, the invention is a method of reducing arterial wave reflections in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In one embodiment, the invention is a method of increasing the concentration of nitrate in plasma in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In another embodiment, the invention is a method of increasing the concentration of nitrite in plasma in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In another embodiment, the invention is a method of improving the vasodilator response to exercise in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In one embodiment, the invention is a method of increasing muscle blood flow during exercise in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In another embodiment, the invention is a method of increasing muscle oxidative capacity in a subject with heart failure, the method comprising administering to the subject a therapeutically effective amount of a composition comprising at least one selected from the group consisting of inorganic nitrate or inorganic nitrite. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot. In various embodiments, the therapeutically effective amount of a composition comprising at least one selected from the group consisting of inorganic nitrate and inorganic nitrite is from about 0.001 mg/kg to about 5 mg/kg. In some embodiments, the composition comprising at least one selected from the group consisting of inorganic nitrate and inorganic nitrite is administered in combination with at least one other agent useful for treating or preventing HFpEF. In various embodiments, the at least one other agent is selected from the group consisting of a diuretic, an angiotensin converting enzyme (ACE) inhibitor, an angiotensin II receptor blocker (ARB), a beta-blocker, a calcium-channel blocker, a statin, an organic nitrate and an organic nitrite. In some embodiments, the subject is human.

In one embodiment, the invention is a composition comprising at least one selected from the group consisting of inorganic nitrate or inorganic nitrite for the treatment or prevention of heart failure in a subject in need thereof. In some embodiments, the heart failure is heart failure with preserved ejection fraction (HFpEF). In some embodiments, the composition is a liquid comprising at least a part of at least one nitrate-containing vegetable. In one embodiment, the nitrate-containing vegetable is beetroot.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a graph depicting how inorganic nitrates/nitrites can target mechanisms proposed to contribute to exercise intolerance in HFPEF.

FIG. 2 is a graph depicting hazard curves for incidence of HF among 5,958 Multi-Ethnic Study of Atherosclerosis (MESA) participants stratified according to the presence or absence of hypertension (prevalence=45%) or the presence or absence of “high” reflection magnitude (top 45% of the population). Curves are adjusted for other significant predictors of HF in this population.

FIG. 2 is a flowchart depicting various effects of the treatments described herein.

### DETAILED DESCRIPTION

The present invention relates to the discovery that dietary inorganic nitrate, or inorganic nitrite (which can be administered, for example, orally or intravenously), is an effective therapy for improving exercise tolerance, symptoms, quality of life, and/or long-term outcomes in patients with heart failure (HF), including HF with preserved ejection fraction (HFpEF). Thus, the invention relates to compositions and methods for treating or preventing HFpEF in a subject by administering a composition comprising at least one selected from the group consisting of inorganic nitrate or inorganic nitrite. Generally, the present invention relates to the discovery that administering inorganic nitrates to a subject is an effective method of treating HFpEF. In some embodiments, the composition comprising at least one selected from the group consisting of inorganic nitrate or inorganic nitrite, is a liquid comprising at least a part of at least one nitrate-containing vegetable. In some embodiments, the nitrate-containing vegetable is beetroot. The invention also relates to the discovery that beetroot and/or sodium nitrite modify key peripheral mechanistic targets in patients with HFpEF, providing both short-term symptom improvement and long-term disease modifying effects. In some embodiments, the invention provides compositions and methods for modulating these mechanistic targets in a subject diagnosed with HFpEF.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±30%-±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “therapeutic” treatment is a treatment administered to a subject who exhibits signs and/or symptoms of a disease or disorder, for the purpose of diminishing or eliminating those signs and/or symptoms.

As used herein, “treating a disease or disorder” means reducing the severity and/or frequency with which a sign and/or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder associated with heart failure, including heart failure with preserved ejection fraction, including alleviating the signs and/or symptoms of such diseases and disorders.

As used herein, the terms “congestive heart failure, (CHF)” “chronic heart failure,” “acute heart failure,” and “heart failure” are used interchangeably, and refer to any condition in which the heart is unable to pump blood at an adequate rate or to do so only in the presence of increased left ventricular filling pressures. When the heart is unable to adequately pump blood to the rest of the body at normal filling left ventricular pressures, blood can back up into the lungs, causing the lungs to become congested with fluid. Typical symptoms of heart failure include shortness of breath (dyspnea), fatigue, weakness, difficulty breathing when lying flat, and swelling of the legs, ankles or abdomen (edema). Causes of heart failure are related to various disorders including coronary artery disease, systemic hypertension, cardiomyopathy or myocarditis, congenital heart disease, abnormal heart valves or valvular heart disease, severe lung disease, diabetes, severe anemia hyperthyroidism, arrhythmia or dysrhythmia and myocardial infarction. Heart failure can occur in the presence of a normal (≧50%) or a reduced (<50%) left ventricular ejection fraction. There is increased recognition that these two conditions represent two different disease states, rather than a continuum (Borlaug B A, Redfield M M. Circulation. 2011 May 10; 123(18):2006-13). HFpEF usually occurs in older patients with risk factors such as obesity, diabetes and hypertension and is more common in women.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the discovery that the administration of nitrate or nitrite is an effective dietary therapy for improving exercise tolerance, symptoms, quality of life and/or long-term outcomes in patients with HF, including HFpEF. Thus, the invention relates to compositions and methods for treating or preventing HF in a subject by administering a composition comprising at least one selected from the group consisting of inorganic nitrate or inorganic nitrite. In some embodiments, the HF is HFpEF. Generally, the present invention relates to the discovery that administering inorganic nitrates or nitrites to a subject through diet is an effective method of treating HFpEF. In some embodiments, the composition comprising at least one selected from the group consisting of inorganic nitrate or inorganic nitrite is a liquid comprising at least a part of at least one nitrate-containing vegetable. In some embodiments, the nitrate-containing vegetable is beetroot.

The invention also relates to the discovery that nitrate modifies key peripheral mechanistic targets in patients with HFpEF, providing both short-term symptom improvement and long-term disease modifying effects. In some embodiments, the invention provides compositions and methods for modulating these peripheral mechanistic targets in a subject diagnosed with HFpEF.

The methods of the present invention are related to the treatment and prevention of HF through the administration of at least one inorganic nitrate or inorganic nitrite. HF is any condition characterized by abnormally low cardiac output in which the heart is unable to pump blood at an adequate rate or does so only in the presence of increased left ventricular filling pressures. In HFpEF, cardiac output at rest is usually preserved, but increased left ventricular filling pressures are present either at rest or during exercise. The present invention provides compositions and methods related to the treatment and prevention of any condition which can be characterized as HF. HF can include a wide variety of symptoms treatable with the compositions and methods of the invention. In some embodiments, the HF comprises impaired left ventricular ejection fraction (“systolic” heart failure). In other embodiments, the HF is preserved ejection fraction (HFpEF, previously called “diastolic” heart failure). Patients with HFpEF have a relatively normal, or near normal, left ventricular ejection fraction (>50%). HFpEF is now seen a complex multi-organ disease that includes peripheral abnormalities in combination with cardiac abnormalities. The present invention provides compositions and methods for modulating these peripheral abnormalities in a subject diagnosed with HFpEF. In various embodiments, the peripheral abnormalities treatable with the compositions and methods of the invention include, but are not limited to, exercise tolerance, vasodilator response, large artery stiffness, and arterial wave deflections.

### Methods

The present invention provides methods for treating or preventing HF by administering a therapeutically effective amount of a composition comprising at least one selected from the group consisting of inorganic nitrite or inorganic nitrate to a subject. Examples of inorganic nitrates include, but are not limited to, sodium nitrate, lithium nitrate, potassium nitrate, cesium nitrate, barium nitrate, and ammonium nitrate. Examples of inorganic nitrites include, but are not limited to, sodium nitrite, lithium nitrite, potassium nitrite, cesium nitrite, and ammonium nitrite. In some embodiments, the HF is HFpEF. In some embodiments, the subject is human. The invention is based in part on the discovery that highly concentrated beetroot juice, which contains a high concentration of nitrates, is an effective therapy for improving exercise tolerance, symptoms, and/or quality of life in patients with HFpEF. Other vegetables known to contain high concentrations of nitrates include, but are not limited to, radishes, turnips, celery, spinach, and lettuce. Diet-derived nitrates or orally administered nitrite are an important endothelium-independent source of the potent vasodilator nitric oxide (NO) through the nitrate-nitrite pathway, which is enhanced in the presence of hypoxia, which occurs within exercising muscle. Dietary nitrates also enhance mitochondrial efficiency and decrease the oxygen cost of exercise. Nitrites induce selective arterial vasodilation induced by hypoxemia, and improve the distribution of blood flow towards and within exercising muscle. This increases O₂ supply to the peripheral muscle in HFpEF. Nitrites may also reduce venous return and preload, which can contribute to improved symptoms.

Many of the symptoms of HFpEF do not result solely from cardiac abnormalities, but are manifested from other peripheral abnormalities. Non-limiting examples of peripheral abnormalities include the exercise vasodilator response, increased arterial wave reflections and arterial stiffness. These abnormalities, which lead to an excessive left ventricular workload, can be favorably affected by inorganic nitrates/nitrites in HFpEF. As such, the present invention provides methods of treating or preventing these peripheral abnormalities in a subject with HF, including HFpEF.

In one embodiment, the invention comprises a method of improving exercise tolerance in a subject with HF, such as HFpEF, by administering a composition comprising at least one selected from the group consisting of inorganic nitrate or inorganic nitrite. As used herein, “exercise tolerance” refers to performing exercises at the level that would be expected of one in their general physical condition or the quantitative performance during a standardized exercise tests (such as the 6-minute walk test or a formal cardiopulmonary stress test). Patients with HFpEF are found to suffer from poor exercise tolerance, resulting in a severely reduced quality of life. During exercise, patients with HFpEF demonstrate impaired vasodilatory responses and a depressed peak oxygen uptake (VO₂). As would be understood by the skilled artisan, measurements for determining exercise tolerance may be acquired through any method known in the art. For example, the distance walked during a standardized 6-minute walk test is a good quantitative surrogate of exercise capacity (Brooks et al., Am J Respir Crit Care Med. 167:1287). In addition, gas analysis during a maximal effort supine-bicycle exercise test may provide parameters to determine peak oxygen consumption (VO₂) and exercise efficiency, as would be understood by one skilled in the art. As used herein, “exercise efficiency” refers to the external power output per amount of oxygen consumed.

In one embodiment, the method of the present invention comprises improving the vasodilator response to exercise in a subject with HF, such as HFpEF, by administering to the subject a composition comprising at least one selected from the group consisting of inorganic nitrate or inorganic nitrite. As would be understood by the skilled artisan, measurements for determining the vasodilator response to exercise may be acquired through any method known in the art. For example, the vasodilator response to exercise may be measured as the change in systemic vascular resistance during a maximal effort supine-bicycle exercise, as would be understood by one skilled in the art.

In one embodiment, the method of the present invention comprises reducing large artery stiffness in a subject with HF, such as HFpEF, by administering to the subject a composition comprising at least one selected from the group consisting of inorganic nitrate or inorganic nitrite. Arterial stiffness is known to increase pulsatile LV afterload in patients with HFpEF. As would be understood by the skilled artisan, measurements for determining large artery stiffness may be acquired through any method known in the art. For example, large artery stiffness may be measured using carotid-femoral pulse wave velocity, an index of aortic stiffness, which is assessed using arterial tonometry or Doppler ultrasound, as would be understood by one skilled in the art.

In one embodiment, the method of the present invention comprises reducing arterial wave reflections in a subject with HF, such as HFpEF, by administering to the subject a composition comprising at least one selected from the group consisting of inorganic nitrate or inorganic nitrite. Arterial wave reflections have been linked to left ventricular remodeling, diastolic dysfunction, and an increased risk of HF. As would be understood by the skilled artisan, measurements for determining arterial wave reflections may be acquired through any method known in the art. For example, arterial wave reflections may be measured by the arterial wave reflection magnitude or augmentation index, which is assessed through analyses of aortic pressure-flow relations using arterial tonometry and Doppler echocardiography, as would be understood by one skilled in the art.

In one embodiment, the method of the present invention comprises increasing muscle blood flow in a subject with HF, such as HFpEF, by administering to the subject a composition comprising at least one selected from the group consisting of inorganic nitrate or inorganic nitrite. As would be understood by the skilled artisan, measurements for determining muscle blood flow may be acquired through any method known in the art. Examples include, but are not limited to, a standardized plantar flexor exercise test or a supine bicycle exercise test, with lower extremity muscle perfusion assessed with arterial MRI spin labeling, femoral Doppler ultrasound or near-infrared spectroscopy, as would be understood by one skilled in the art. As used herein, “vascular resistance” refers to the ratio of mean arterial pressure/blood flow.

In one embodiment, the method of the present invention comprises decreasing vascular resistance and increasing skeletal muscle blood flow in a subject with HF, such as HFpEF, by administering to the subject a composition comprising a nitrate or nitrite. As would be understood by the skilled artisan, measurements for determining skeletal muscle blood flow may be acquired through any method known in the art. Examples include, but are not limited to a cuff occlusion test in which muscle perfusion is assessed with Doppler ultrasound or near-infrared spectroscopy. As used herein, “vascular resistance” refers to the ratio of mean arterial pressure/blood flow.

In one embodiment, the method of the present invention comprises increasing muscle oxidative capacity in a subject with HF, such as HFpEF, by administering to the subject a composition comprising at least one selected from the group consisting of inorganic nitrate or inorganic nitrite. As would be understood by the skilled artisan, measurements for determining the vasodilator response to exercise may be acquired through any method known in the art. Examples include, but are not limited to, a standardized plantar flexor exercise test, muscle phosphocreatine (PCr) kinetics measured with phosphorus spectroscopy, chemical exchange saturation transfer which allows for imaging of PCr concentrations, or near infrared spectroscopy measurements of muscle O₂ consumption immediately after mild exercise using transient arterial occlusions, as would be understood by one skilled in the art.

In one embodiment, the method of the present invention comprises reducing preload in a subject with HF, such as HFpEF, by administering to the subject a composition comprising at least one selected from the group consisting of inorganic nitrate or inorganic nitrite. As would be understood by the skilled artisan, measurements for determining preload would include the measurement of left ventricular end-diastolic pressure with a catheter, pulmonary capillary wedge pressure with a catheter, or indices of diastolic mitral filling with Doppler echocardiography, as would be understood by one skilled in the art.

Various embodiments of the methods of the invention comprise administering a therapeutically effective amount of a composition comprising at least one of inorganic nitrate or inorganic nitrite. In some embodiments, the therapeutically effective amount of a composition comprising at least one of inorganic nitrate or inorganic nitrite is between 0.1 to 100 mmol of inorganic nitrates, organic nitrates, inorganic nitrites, or organic nitrates. In other embodiments, the therapeutically effective amount of a composition comprising at least one of inorganic nitrate or inorganic nitrite is between 1 to 50 mmol of nitrates. In other embodiments, the therapeutically effective amount of a composition comprising at least one of inorganic nitrate or inorganic nitrite is between 5 to 25 mmol of inorganic nitrates or inorganic nitrites. In further embodiments, the therapeutically effective amount of a composition comprising at least one of inorganic nitrate or inorganic nitrite is between 10 to 15 mmol of inorganic nitrates or inorganic nitrites.

In some embodiments, the composition comprising at least one of inorganic nitrate or inorganic nitrite is comprised of a therapeutically effective amount of sodium nitrite. In some embodiments, the therapeutically effective amount of sodium nitrite is between 0.01 mg and 1000 mg. In other embodiments, the therapeutically effective amount of sodium nitrite is between 1 mg and 500 mg. In other embodiments, the therapeutically effective amount of sodium nitrite is between 10 mg and 100 mg. In one embodiment, the therapeutically effective amount of sodium nitrite is 80 mg.

Additionally, as disclosed elsewhere herein, one skilled in the art would understand, once armed with the teaching provided herein, that the present invention encompasses a method of preventing a wide variety of diseases, disorders and pathologies where administration of at least one inorganic nitrate or inorganic nitrite treats or prevents the disease, disorder or pathology. Methods for assessing whether a disease relates to diminished levels of an inorganic nitrate or an inorganic nitrite are known in the art. Further, the invention encompasses treatment or prevention of such diseases discovered in the future.

The invention encompasses administration of a composition comprising at least one inorganic nitrate or inorganic nitrite to practice the methods of the invention; the skilled artisan would understand, based on the disclosure provided herein, how to formulate and administer the appropriate composition comprising at least one selected from the group consisting of inorganic nitrate or inorganic nitrite to a subject. Indeed, the successful administration of the composition comprising at least one selected from the group consisting of inorganic nitrate or inorganic nitrite has been reduced to practice as exemplified herein. However, the present invention is not limited to any particular method of administration or treatment regimen.

### Compositions

The present invention provides compositions comprised of at least one inorganic nitrate or inorganic nitrite for the treatment and prevention of HF, including HFpEF. Any composition comprising at least one selected from the group consisting of inorganic nitrate or inorganic nitrite is contemplated by the present invention. Examples of inorganic nitrates include, but are not limited to, sodium nitrate, lithium nitrate, potassium nitrate, cesium nitrate, barium nitrate, and ammonium nitrate. Examples of organic nitrates include, but are not limited to, dialkyl imidazolium nitrates, and guanidine nitrate. Examples of inorganic nitrites include, but are not limited to, sodium nitrite, lithium nitrite, potassium nitrite, cesium nitrite, and ammonium nitrite. Examples of organic nitrites include, but are not limited to, ethyl nitrite, propyl nitrite, butyl nitrite, pentyl nitrite, and octyl nitrite. The composition comprising at least one selected from the group consisting of inorganic nitrate or inorganic nitrite may comprise any form, as would be understood by one skilled in the art. Non-limiting examples of forms include a liquid, a paste, a gel, a bar, a cake, a powder, a granulate, an effervescent tablet, a chewing gum, a tablet, a capsule, a lozenge, a fast melting tablet or wafer, a sublingual tablet or a spray. Such products can be manufactured using conventional methods practiced in the food and beverage industry, or in pharmaceutical industry.

Preferably, the composition comprising at least one selected from the group consisting of inorganic nitrate or inorganic nitrite is a liquid comprising at least a part of at least one nitrate-containing vegetable. Vegetables are known to be an important source of nitrates in the diet. Examples of vegetables rich in nitrates are green leafy vegetables, spinach, beetroot, fennel, lettuce, cabbage and the like. Juices, pastes, concentrates, and other such compositions of such vegetables are contemplated as suitable sources of nitrate. As contemplated herein, any nitrate-containing vegetable may be used, either separately or in any combination, and in any concentration, in the creation of compositions comprising at least one inorganic nitrate or inorganic nitrite of the present invention. In one embodiment, the nitrate-containing vegetable is beetroot. The liquid comprising at least a part of at least one nitrate-containing vegetable can be prepared by any method known in the art. By way of example, the liquid can be prepared by placing the vegetable in a press and collecting the released juices. By way of another example, the vegetable can be prepared by placing the vegetable in a blender and collecting the blended vegetable.

Compositions identified as potentially useful compounds containing at least one inorganic nitrate or inorganic nitrite for the treatment and/or prevention of heart disease, such as HFpEF, can be formulated and administered to a subject for treatment or prevention of heart disease, such as HFpEF, as now described.

The invention encompasses the preparation and use of compositions comprising a composition useful for treatment of heart disease, such as HFpEF, disclosed herein as a composition comprising at least one selected from the group consisting of inorganic nitrate or inorganic nitrite. Such a composition may consist of the at least one inorganic nitrate or inorganic nitrite alone, in a form suitable for administration to a subject, or the composition may comprise the at least one inorganic nitrate or inorganic nitrite and one or more pharmaceutically acceptable carriers, one or more additional ingredients, or some combination of these. The at least one inorganic nitrate or inorganic nitrite may be present in the composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor thereof, may be combined and which, following the combination, can be used to administer the appropriate inhibitor thereof, to a subject.

The compositions useful for practicing the invention may be administered to deliver a dose of nitrate and/or nitrite between about 0.1 ng/kg/day and 100 mg/kg/day.

In various embodiments, the compositions useful in the methods of the invention may be administered, by way of example, systemically or parenterally, such as, in oral formulations. In addition to the appropriate therapeutic composition, such compositions may contain pharmaceutically acceptable carriers and other ingredients known to enhance and facilitate drug administration.

As used herein, the term “physiologically acceptable” ester or salt means an ester or salt form of the at least one inorganic nitrate or inorganic nitrite which is compatible with any other ingredients of the composition, which is not deleterious to the subject to which the composition is to be administered.

The formulations of the compositions described herein may be prepared by any method known or hereafter developed in the art of pharmacology. In general, such preparatory methods include the step of bringing the at least one inorganic nitrate or inorganic nitrite into association with a carrier or one or more other accessory ingredients, and then, if necessary or desirable, shaping or packaging the product into a desired single- or multi-dose unit.

Although the descriptions of compositions provided herein are principally directed to compositions which are suitable for ethical administration to humans, it will be understood by the skilled artisan that such compositions are generally suitable for administration to animals of all sorts. Modification of compositions suitable for administration to humans in order to render the compositions suitable for administration to various animals is well understood, and the ordinarily skilled veterinary pharmacologist can design and perform such modification with merely ordinary, if any, experimentation.

Compositions that are useful in the methods of the invention may be prepared, packaged, or sold in formulations suitable for oral, parenteral, intravenous, and other known routes of administration.

A composition of the invention may be prepared, packaged, or sold in bulk, as a single unit dose, or as a plurality of single unit doses. As used herein, a “unit dose” is discrete amount of the composition comprising a predetermined amount of the nitrate. The amount of the nitrate is generally equal to the dosage of at least one inorganic nitrate or inorganic nitrite which would be administered to a subject or a convenient fraction of such a dosage such as, for example, one-half or one-third of such a dosage.

The relative amounts of the at least one inorganic nitrate or inorganic nitrite, the pharmaceutically acceptable carrier, and any additional ingredients in a composition of the invention will vary, depending upon the identity, size, and condition of the subject treated and further depending upon the route by which the composition is to be administered. By way of example, the composition may comprise between 0.1% and 100% (w/w) nitrate.

In addition to the at least one inorganic nitrate or inorganic nitrite, a composition of the invention may further comprise one or more additional pharmaceutically active agents.

Controlled- or sustained-release formulations of a composition of the invention may be made using conventional technology.

A formulation of a composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the at least one inorganic nitrate or inorganic nitrite. Other formulations suitable for oral administration include, but are not limited to, a powdered or granular formulation, an aqueous or oily suspension, an aqueous or oily solution, or an emulsion.

A tablet comprising the at least one inorganic nitrate or inorganic nitrite may, for example, be made by compressing or molding the at least one inorganic nitrate or inorganic nitrite, optionally with one or more additional ingredients. Compressed tablets may be prepared by compressing, in a suitable device, the at least one inorganic nitrate or inorganic nitrite in a free-flowing form such as a powder or granular preparation, optionally mixed with one or more of a binder, a lubricant, an excipient, a surface active agent, and a dispersing agent. Molded tablets may be made by molding, in a suitable device, a mixture of the at least one inorganic nitrate or inorganic nitrite, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the at least one inorganic nitrate or inorganic nitrite. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the at least one inorganic nitrate or inorganic nitrite may be made using a physiologically degradable composition, such as gelatin. Such hard capsules comprise the at least one inorganic nitrate or inorganic nitrite, and may further comprise additional ingredients including, for example, an inert solid diluent such as calcium carbonate, calcium phosphate, or kaolin.

Soft gelatin capsules comprising the at least one inorganic nitrate or inorganic nitrite may be made using a physiologically degradable composition, such as gelatin. Such soft capsules comprise the at least one inorganic nitrate or inorganic nitrite, which may be mixed with water or an oil medium such as peanut oil, liquid paraffin, or olive oil.

Liquid formulations of a composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the at least one inorganic nitrate or inorganic nitrite in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the at least one inorganic nitrate or inorganic nitrite in aqueous or oily solvents may be prepared in substantially the same manner as liquid suspensions, the primary difference being that the at least one inorganic nitrate or inorganic nitrite is dissolved, rather than suspended in the solvent. Liquid solutions of the composition of the invention may comprise each of the components described with regard to liquid suspensions, it being understood that suspending agents will not necessarily aid dissolution of the at least one inorganic nitrate or inorganic nitrite in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

As used herein, “parenteral administration” of a composition includes any route of administration characterized by physical breaching of a tissue of a subject and administration of the composition through the breach in the tissue. Parenteral administration thus includes, but is not limited to, administration of a composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, and intramuscular, intracisternal injection.

Formulations of a composition suitable for parenteral administration comprise the at least one inorganic nitrate or inorganic nitrite combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the at least one inorganic nitrate or inorganic nitrite is provided in dry (i.e., powder or granular) form for reconstitution with a suitable vehicle (e.g., sterile pyrogen-free water) prior to parenteral administration of the reconstituted composition.

The compositions may be prepared, packaged, or sold in the form of a sterile injectable aqueous or oily suspension or solution. This suspension or solution may be formulated according to the known art, and may comprise, in addition to the at least one inorganic nitrate or inorganic nitrite, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the at least one inorganic nitrate or inorganic nitrite in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A composition of the invention may be prepared, packaged, or sold in a formulation suitable for buccal administration. Such formulations may, for example, be in the form of tablets or lozenges made using conventional methods, and may, for example, contain 0.1 to 100% (w/w) nitrate, the balance comprising an orally dissolvable or degradable composition and, optionally, one or more of the additional ingredients described herein. Alternately, formulations suitable for buccal administration may comprise a powder or an aerosolized or atomized solution or suspension comprising the at least one inorganic nitrate or inorganic nitrite. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to about 100 g per kilogram of body weight of the animal. The precise dosage administered will vary depending upon any number of factors, including, but not limited to, the type of animal and type of disease state being treated, the age of the animal and the route of administration. In some embodiments, the dosage of the compound will vary from about 1 mg to about 100 mg per kilogram of body weight of the animal. In other embodiments, the dosage will vary from about 1 μg to about 1 g per kilogram of body weight of the animal. The compound can be administered to an animal as frequently as two, three, four, five, six, seven or eight times daily, or it can be administered less frequently, such as once a day, one or more times a week, one or more times every two weeks, one or more times a month, or even less frequently, such as one or more times every several months or even one or more times a year. The frequency of the dose will be readily apparent to the skilled artisan and will depend upon any number of factors, such as, but not limited to, the type and severity of the disease being treated, the type and age of the animal, etc.

### Combination Therapy

In some embodiments, the composition comprising at least one selected from the group consisting of inorganic nitrate or inorganic nitrite may be combined with at least one other agent useful for treating or preventing HF, such as HFpEF. Examples of agents useful for treating or preventing HF, such as HFpEF, include, but are not limited to, diuretics, angiotensin converting enzyme (ACE)-inhibitors, angiotensin II receptor blockers (ARBs), beta-blockers, calcium-channel blockers, digoxin, statins, organic nitrate or organic nitrite. Examples of organic nitrates include, but are not limited to, dialkyl imidazolium nitrates, and guanidine nitrate. Examples of organic nitrites include, but are not limited to, ethyl nitrite, propyl nitrite, butyl nitrite, pentyl nitrite, and octyl nitrite.

In one embodiment, an additional therapeutic agent is administered to a subject in combination with a composition comprising at least one selected from the group consisting of inorganic nitrate or inorganic nitrite, such that a synergistic therapeutic effect is produced. A “synergistic therapeutic effect” refers to a greater-than-additive therapeutic effect which is produced by a combination of two therapeutic agents, and which exceeds that which would otherwise result from individual administration of either therapeutic agent alone. Therefore, lower doses of one or both of the therapeutic agents may be used for treating or preventing HF, such as HFpEF, resulting in increased therapeutic efficacy and decreased side-effects. In some embodiments, the agent is a phosphodiesterase 5 (PED5) inhibitor or an organic nitrate. Examples of PED5 inhibitors include, but are not limited to, sildenafil, vardenafil, and tadalafil.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Use of Inorganic Nitrate in Heart Failure (HF) with Preserved Ejection Fraction

Described herein is a dietary intervention targeted at specific mechanisms likely to play a role in exercise intolerance in HFpEF. This is a novel dietary treatment (inorganic nitrate supplementation) for the modification of key peripheral mechanistic targets (e.g., arterial vasodilator reserve, muscle O₂ delivery and utilization, arterial wave reflections and arterial stiffness), which has the potential for both short-term symptom-improvement and long-term “disease-modifying” effects of HFpEF patients. This dietary treatment represents a new therapeutic paradigm and can provide a readily implementable approach on improving symptoms, exercise capacity and outcomes in HFpEF.

Described herein is a study in which 22 subjects with HFpEF are randomized, in a double-blind cross-over design, and assigned to a single dose of 140 mL of: (a) Nitrate-rich concentrated beetroot juice (NO₃⁻_{RICH}BR, containing 12.9 mmol of NO⁻₃), or; (b) An otherwise identical, nitrate-depleted concentrated beetroot juice (NO₃⁻ DEP BR, containing <0.01 mmol of NO⁻₃). Supplementation with NO₃⁻_{RICH} BR is examined for improvements of the following endpoints: exercise performance, the exercise systemic vasodilator reserve and, more specifically, the vasodilator response in working muscle, muscle oxidative capacity, arterial wave reflections and large artery stiffness.

### Late Systolic Load Promotes Diastolic Dysfunction

The time course of systolic left ventricular (LV) wall stress in humans (Chirinos et al., 2009, Circulation 119:2798-2807), allowing the separation of early and late systolic wall stress, quantified as the area under the time-resolved stress curve (stress-time integral, STI) in the first and second halves of ejection, respectively. Using this technique, the relationship between the myocardial loading sequence (early vs. late stress) and diastolic function was assessed among 1,215 middle-aged adults enrolled in the Asklepios study (Chirinos et al., 2009, Circulation 119:2798-2807; Chirinos et al., 2009, Hypertension 54:558-566; Chirinos et al., 2010, Hypertension 56:91-98). After adjustment for confounders, late systolic load was associated with lower mitral annular velocity (an index of LV relaxation), in contrast to early systolic load (which was associated with higher relaxation velocities in a multivariate model that predicted 46% of the variability in mitral annular relaxation velocity, Table 1). Available evidence thus implicates the loading sequence as an independent correlate of LV relaxation in humans.

TABLE 1
Early and Late Systolic Stress as Predictors of Early Diastolic
Mitral Annular Velocity in a Multivariate Model (R² =
0.46) among 1,215 Adults in the Asklepios Study

| | Standardized | |
|---|---|---|
| | Coefficient | |
| Independent variables | β | P value |
| (Constant) | | <0.0001 |
| Late ejection-phase STI (kdynes · cm⁻² · s) | −0.25 | <0.0001 |
| Early ejection-phase STI (kdynes · cm⁻² · s) | 0.18 | <0.0001 |
| Age (years) | −0.34 | <0.0001 |
| Male gender | −0.17 | <0.0001 |
| Body height (m) | 0.068 | 0.06 |
| Body weight (kg) | −0.38 | <0.0001 |
| Total cholesterol (mg/dl) | −0.067 | 0.005 |
| HDL-cholesterol (mg/dl) | 0.073 | 0.008 |
| Triglycerides (mg/dl) | −0.027 | 0.27 |
| Estimated GFR, mL · min⁻¹ · 1.73 m⁻² | −0.008 | 0.71 |
| High-sensitive CRP (In-transformed; mg/dl) | −0.002 | 0.91 |
| Current smoking | 0.025 | 0.26 |
| Diabetes meilitus | 0.005 | 0.84 |
| LV sphericity | 0.14 | <0.0001 |
| Antihypertensive medication use | −0.027 | 0.23 |
| Heart rate (bpm) | −0.017 | 0.47 |

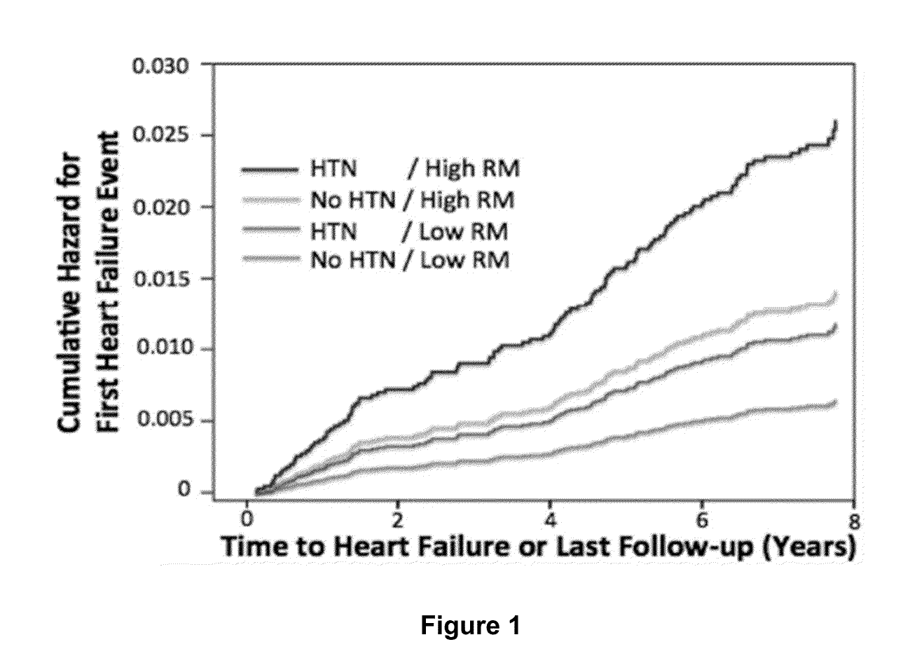

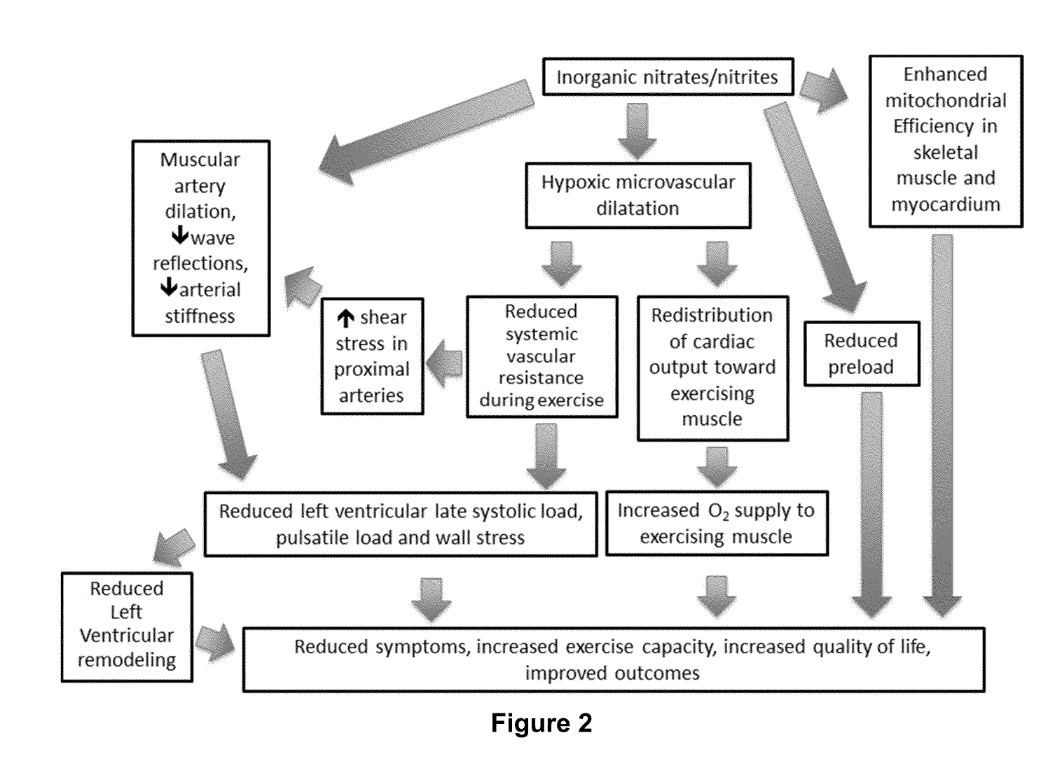

The magnitude of wave reflections strongly predicts incident HF. Based on the data presented above, and without wishing to be bound by any particular theory, it was hypothesized that wave reflections independently predict the risk of new-onset HF in the general population. Aortic pressure waveforms were derived using a transfer function applied to the radial waveform recorded at baseline with arterial tonometry from 5,934 participants in the Multiethnic Study of Atherosclerosis (MESA), who were free of clinically apparent cardiovascular disease. The central pressure waveform was used to approximate reflection magnitude as previously described (Westerhof et al., 1972, Cardiovasc. Res. 6:648-656). During 7.61 years of follow-up (and after adjustment for blood pressure, age, gender, body mass index, diabetes, ethnicity, antihypertensive medication use, total and HDL-cholesterol, current smoking, heart rate and glomerular filtration rate), reflection magnitude strongly predicted HF (Hazard ratio per 10%−increase=2.69; 95% CI=1.79-4.04; P<0.0001) and was a stronger predictor of HF than blood pressure and other modifiable risk factors listed above. In a model that adjusted for other HF predictors, compared to non-hypertensive subjects with low reflection magnitude, hazard ratios for hypertensive subjects with low reflection magnitude, non-hypertensive subjects with high reflection magnitude and hypertensive subjects with high reflection magnitude were 1.81 (95% CI=0.85-3.86), 2.16 (95% CI=1.04-4.43) and 3.98 (95% CI=1.96-8.05), respectively (FIG. 1). Without wishing to be bound by any particular theory, these findings from a large community-based sample with careful follow-up and event adjudication, are consistent with the explanation that arterial wave reflections are a novel strong risk factor for HF.

### Dietary Nitrates Reduce Wave Reflections

Since NO-mediated vasodilation of middle-sized muscular conduit arteries (Nichols and Vlachopolous, 2011, McDonald's blood flow in arteries. Theoretical, experimental and clinical principles, Hodder Arnold; O-Rourke and Hashimoto, 2007, J. Am. College Cardiol. 50:1-13; Yaginuma et al., 1986, Cardiovasc. Res. 20:153-160; Kelly et al., 1990, Eur. Heart J. 11:138-144; Latson et al., 1988, Circ. Res. 62:884-890) can substantially reduce wave reflections and wave reflections lead to myocardial dysfunction, experiments were designed to target wave reflections in patients with HFpEF using inorganic nitrates/nitrites. Without wishing to be bound by any particular theory, inorganic nitrates/nitrites may lead to NO release in middle muscular arteries, which would reduce wave reflections and/or may increase distal blood flow through microvascular dilation, which would increase the shear stress on more proximal vessels, leading to flow-mediated dilation. These effects would lead to reduced wave reflections.

### Approach

22 subjects with HFpEF are assigned to 140 mL/day of either: (a) Nitrate-rich concentrated beetroot juice (NO₃⁻_{RICH}BR, containing 12.9 mmol of NO⁻³), or; (b) an otherwise identical, nitrate-depleted beetroot juice (NO₃⁻_{DEPL}BR, containing <0.01 mmol of NO⁻³) for 3 days. The study is double blind and cross-over controlled. The order of the interventions is randomized, with a 7-day washout period separating each supplementation period. A crossover design enables each subject to receive both treatments, reducing inter-individual response variability. NO₃⁻_{RICH}BR and NO₃⁻ is provided by James White Drinks Ltd., (Ipswich, United Kingdom, U.K.). NO₃⁻_{RICH}BR and NO₃⁻_{DEPL} are dispensed by an investigational drug pharmacist. All study procedures are double-blinded. Subjects are instructed to keep a stable intake of vegetables and to avoid antibacterial mouthwash throughout the supplementation period. Depletion of nitrates for the control juice is achieved using an ion-exchange resin that selectively removes nitrate (Lansley et al., 2011, J. Appl. Physiol. 110:591-600), resulting in a juice otherwise similar in appearance, odor, taste, and texture, allowing assessment of whether dietary nitrates are responsible for the postulated effects and to implement a double-blind experimental design. An intervention-related change in plasma nitrates/nitrites is documented. Without wishing to be bound by any particular theory, it is hypothesized that supplementation with NO₃⁻_{RICH}BR improves exercise performance, the exercise systemic vasodilator reserve and the vasodilator response in working muscle, muscle oxidative capacity, and arterial stiffness and wave reflections.

### Study Population

Inclusion Criteria

22 adults are enrolled. These adults have HFpEF and New York Heart Association Class II-IV symptoms, LV ejection fraction >50%, stable medical therapy (no addition, removal or dose change by of anti-hypertensive agents or diuretics for at least 30 days), and evidence of significant diastolic dysfunction, thus meeting European Society of Echocardiography criteria for the diagnosis of HfpEF (Paulus et al., 2007, Eur. Heart J. 28:2539-2550).

Exclusion Criteria

An adult having any of the following criteria is excluded: atrial fibrillation or flutter, neuromuscular or orthopedic condition that prevents subject from exercising, more than mild valvular heart disease, hypertrophic, infiltrative or inflammatory cardiomyopathy, pericardial disease, primary pulmonary arteriopathy, acute coronary syndrome or coronary revascularization within 60 days, more than mild obstructive lung disease, non-revascularized significant myocardial ischemia on a stress test within 1 year, allergy to beetroot, therapy with phosphodiesterase inhibitors, or contraindications or unwillingness to undergo an MRI study.

### Endpoints

Endpoints are measured before the first dose of either NO₃⁻_{RICH}BR or NO₃⁻_{DEPL}BR and at the end of the double-blinded 3-day supplementation period with either NO₃⁻_{DEPL}BR or NO₃⁻_{RICH}BR phase.

### Exercise Capacity

Exercise capacity is measured with a cardiopulmonary stress test during supine bicycle exercise. Peak oxygen consumption (VO₂) and [peak external power output/peak VO₂] ratio are assessed via expired gas analysis during a maximal effort supine cycle exercise test followed by a constant intensity submaximal exercise protocol below the ventilatory threshold to achieve steady state oxygen consumption. Expired gas analyses are made using a Parvomedics TrueOne device. Gas meter and flow sensor calibration are performed before each test. Beta-blockers are withheld for at least 48 h prior to testing.

### Systemic Arterial Hemodynamics

A high-fidelity Millar applanation tonometer (Nichols and Vlachopolous, 2011, Mcdonald's blood flow in arteries. Theoretical, experimental and clinical principles, Hodder Arnold) is used to record carotid pressure waveforms, which are calibrated with using brachial diastolic and mean pressures measured with a validated oscillometric device (Segers et al., 2007, Hypertension 49:1248-1255). Doppler echocardiography is performed using a Vivid E9 device. Pulsed-wave Doppler interrogation of LV outflow tract flow velocities is performed at rest and peak exercise. Flow volume is computed by multiplying LV outflow tract flow velocity by LV outflow tract cross-sectional area measured with 3D echocardiography (Chirinos and Segers, 2010, Hypertension 56:563-570; Chirinos and Segers, 2010, Hypertension 56:555-562). Reflection magnitude is computed using linear wave separation analysis using central pressure and flow waveforms (Chirinos and Segers, 2010, Hypertension 56:563-570; Chirinos and Segers, 2010, Hypertension 56:555-562; Segers et al., 2007, Hypertension 49:1248-1255; Westerhof et al., 1972, Cardiovasc. Res. 6:648-656). Carotid-femoral pulse wave velocity is measured with arterial tonometry (Sphygmocor device, Atcor Medical). Augmentation index, which is the ratio of the second to first systolic peak, is also assessed. For exercise hemodynamics, arterial pressure at peak exercise is measured using a validated photoplethysmographic device (Finapress device). Systemic vascular resistance (SVR) is computed as [mean arterial pressure/cardiac output]. Exercise vasodilatory reserve is computed as rest SVR minus exercise SVR.

### Muscle Perfusion and Energetics

MRI studies are performed at rest and immediately after a standardized plantar flexion exercise test using a 7T scanner equipped with a 28-channel radiofrequency coil. Arterial spin labeling (Roberts et al., 1994, Proc. Natl. Acad. Sci. USA 91:33-37) is used to image muscle perfusion. ³¹P magnetic resonance spectroscopy is used to study phosphocreatine (PCr) recovery kinetics following exercise. Intracellular pH is calculated from the chemical shift difference between inorganic phosphate (Pi) and PCr (Moon and Richards, 1973, J. Biol. Chem. 248:7276-7278), which is used to calculate free cytosolic ADP using the creatine kinase (CK) equilibrium constant (Kemp et al., 2001, J. Physiol. 535:910-928). Changes in pH and in the concentration of phosphorus metabolites are used to calculate oxidative capacity and the rates of ATP synthesis through the CK reaction, oxidative phosphorylation, and anaerobic glycolysis as previously described (Kemp et al., 1994, Magn. Reson. Q. 10:43-63; Trenell et al., 2006, Muscle Nerve 33:524-531; Conley et al., 1997, Am. J. Physiol. 273:C306-315; Layec et al., 2009, Eur. J. Appl. Physiol. 106:229-242). The correlation between PCr recovery kinetics and muscle perfusion is assessed. The phosphocreatine content of skeletal muscle is imaged using chemical exchange saturation transfer methods (Cai et al., 2012, Nat. Med. 18:302-306; Singh et al., 2011, Int. Soc. Mag. Res. Med. 19:4619). Correlation between perfusion and PCr recovery kinetics is assessed using voxel-wise correlation analyses.

### Nitrate and Nitrite Level Measurements

Venous blood samples are drawn into lithium-heparin tubes, which have very low levels of nitrate/nitrite. Samples are centrifuged at 4,000 rpm for 10 min, within 3 min of collection. Plasma is extracted and immediately frozen at −80° C. for later analysis. After thawing at room temperature, plasma samples are initially deproteinized using cold ethanol precipitation as previously described (Lansley et al., 2011, J. Appl. Physiol. 110:591-600). The nitrate/nitrite content of deproteinized plasma is determined using a modified detection chemiluminescence technique using a Ionics/Sievers nitric oxide analyzer (NOA 280), as elsewhere described (see Munson et al., 2005, Am. J. Respir. Cell Mol. Biol. 33:582-588) and later adapted by Allen et al for human plasma (Allen et al., 2010, Free Radic. Biol. Med. 49:1138-1144).

### Statistical Power and Methods

22 subjects are randomized to one of 2 sequences, each of which consists of 2 periods (AB/BA design). The study has 80% power to detect standardized differences of 0.549 or greater in the intervention-induced change of endpoints, with a one-sided α=0.05. For inferential analyses, the general model for crossover with continuous data is followed:

Yᵢ₍ⱼ₎ₖ=αᵢₖ+sᵢ₍ⱼ₎+εᵢ₍ⱼ₎ₖ,
where Yᵢ₍ⱼ₎ₖ is the observed outcome, sᵢ₍ⱼ₎ is a an effect due to subject j of sequence i, j=1, 2, . . . 22, αᵢₖ is an effect indexed by sequence i and period k and εᵢ₍ⱼ₎ₖ is a random “error” term with expectation 0 and variance γ². It follows that αᵢₖ=E[Yᵢ₍ⱼ₎ₖ]−E[sᵢ₍ⱼ₎)]. Interest centers around αᵢₖ which can be expressed in terms of treatment, period and possibly carryover effects: αᵢₖ=μ+τ_{d(i,k)}+πₖ+λ_{d(i,k-1)}, where τ_{d(i,k)} is the effect of the treatment in period k from sequence I, irₖ is the effect of period k, +λ_{d(i,k-1)} is a carryover effect arising from treatment in period (k−1) from sequence i, which will change the effect of treatment in period k from sequence i.

The statistical tests used are based on the distribution of the outcome. With normality, for each sequence the average of the difference of the second period from the first is calculated, allowing for computing the difference of these two averages as a good unbiased estimate of the treatment effect. The period effect drops out using these differences. An (unpaired) t-test can be used to assess the difference. For each sequence, the average of the difference of the two periods is calculated, allowing computation of the sum of these two averages as a good unbiased estimate of the period effect difference. An (unpaired) t-test can be used to assess the effect by multiplying the differences for one sequence by −1, so that the two average differences are essentially summed. In the case of non-normal distributed outcomes, non-parametric methods are utilized. Generalized linear mixed models are employed to assess the findings in a regression framework, with additional adjustment for covariates as needed.

### Example 2

### Use of Sodium Nitrite in Heart Failure (HF) with Preserved Ejection Fraction

Described herein is a pharmacologic intervention targeted at specific mechanisms likely to play a role in exercise intolerance in HFpEF using sodium nitrite, an inorganic nitrite. This is a novel pharmacologic treatment for the modification of key peripheral mechanistic targets (e.g., arterial vasodilator reserve, muscle O₂ delivery and utilization, arterial wave reflections and arterial stiffness), which has the potential for both short-term symptom-improvement and long-term “disease-modifying” effects of HFpEF patients. This treatment represents a new therapeutic paradigm and can provide a readily implementable approach on improving symptoms, exercise capacity and outcomes in HFpEF.

Described herein is a study in which 76 subjects with HFpEF are randomized, in a double-blind cross-over design, and assigned to; (1) sodium nitrite administered orally for 4-6 weeks, or; (b) an otherwise identical placebo. The sequence of interventions is randomized, double-blind and separated by a 7-day washout period. Supplementation with sodium nitrite is examined for improvements of the following endpoints: exercise performance, the exercise systemic vasodilator reserve and, more specifically, the vasodilator response in working muscle, muscle oxidative capacity, arterial wave reflections, large artery stiffness, quality of life. Exercise performance, the exercise systemic vasodilator reserve and, more specifically, the vasodilator response in working muscle, muscle oxidative capacity, arterial wave reflections, large artery stiffness are assessed using methods similar to those described in Example 1. Quality of life is assessed using the Kansas City Cardiomyopathy Questionnaire (KCCQ) (Green C P, Porter C B, Bresnahan D R, Spertus J A. Development and evaluation of the kansas city cardiomyopathy questionnaire: A new health status measure for heart failure. Journal of the American College of Cardiology. 2000; 35:1245-1255).

### Example 3

### Use of Potassium Nitrate in Heart Failure (HF) with Preserved Ejection Fraction

Described herein is a pharmacologic intervention targeted at specific mechanisms likely to play a role in exercise intolerance in HFpEF using potassium nitrate, an inorganic nitrate. This is a novel pharmacologic treatment for the modification of key peripheral mechanistic targets (e.g., arterial vasodilator reserve, muscle O₂ delivery and utilization, arterial wave reflections and arterial stiffness), which has the potential for both short-term symptom-improvement and long-term “disease-modifying” effects of HFpEF patients. This treatment represents a new therapeutic paradigm and can provide a readily implementable approach on improving symptoms, exercise capacity and outcomes in HFpEF.

Described herein is a study in which subjects with HFpEF are randomized, in a double-blind cross-over design, and assigned to; (1) potassium nitrate administered orally for 4-6 weeks, or; (b) an otherwise identical placebo. The sequence of interventions is randomized, double-blind and separated by a 7-day washout period. Supplementation with potassium nitrate is examined for improvements of the following endpoints: exercise performance, the exercise systemic vasodilator reserve and, more specifically, the vasodilator response in working muscle, muscle oxidative capacity, arterial wave reflections, large artery stiffness, quality of life. Exercise performance, the exercise systemic vasodilator reserve and, more specifically, the vasodilator response in working muscle, muscle oxidative capacity, arterial wave reflections, large artery stiffness are assessed using methods similar to those described in Example 1. Quality of life is assessed using the Kansas City Cardiomyopathy Questionnaire (KCCQ) (Green C P, Porter C B, Bresnahan D R, Spertus J A. Development and evaluation of the kansas city cardiomyopathy questionnaire: A new health status measure for heart failure. Journal of the American College of Cardiology. 2000; 35:1245-1255).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing heart failure in a subject in need thereof, the method comprising administering to the subject a therapeutically effective amount of a composition comprising at least one selected from the group consisting of inorganic nitrate, organic nitrate, inorganic nitrite, and organic nitrite.

2. The method of claim 1, wherein the heart failure is heart failure with preserved ejection fraction (HFpEF).

3. The method of claim 1, wherein the composition comprising at least one selected from the group consisting of inorganic nitrate, organic nitrate, inorganic nitrite, and organic nitrite is a liquid comprising at least a part of at least one nitrate-containing vegetable.

4. The method of claim 3, wherein the at least a part of at least one nitrate-containing vegetable is beetroot.

5. The method of claim 1, wherein the therapeutically effective amount of a composition comprising at least one selected from the group consisting of inorganic nitrate, organic nitrate, inorganic nitrite, and organic nitrite is from about 0.001 mg/kg to about 5 mg/kg.

6. The method of claim 1, wherein the composition comprising at least one selected from the group consisting of inorganic nitrate, organic nitrate, inorganic nitrite, and organic nitrite is administered in combination with at least one other agent useful for treating or preventing HFpEF.

7. The method of claim 6, wherein the at least one other agent is selected from the group consisting of a diuretic, an angiotensin converting enzyme (ACE) inhibitor, an angiotensin 11 receptor blocker (ARB), a beta-blocker, a calcium-channel blocker, digoxin, and a statin.

8. (canceled)

9. A method of improving exercise tolerance, or reducing large artery stiffness, or reducing arterial wave reflections, or improving the vasodilator response to exercise, or increasing muscle blood flow during exercise, or increasing muscle oxidative capacity, in a subject with heart failure, the method comprising administering to the subject a therapeutically effective amount of a composition comprising at least one selected from the group consisting of inorganic nitrate, organic nitrate, inorganic nitrite, or organic nitrite.

10. The method of claim 9, wherein the heart failure is heart failure with preserved ejection fraction (HFpEF).

11. The method of claim 9, wherein the composition comprising at least one selected from the group consisting of inorganic nitrate, organic nitrate, inorganic nitrite, or organic nitrite is a liquid comprising at least a part of at least one nitrate-containing vegetable.

12. The method of claim 11, wherein the at least a part of at least one nitrate-containing vegetable is beetroot.

13-23. (canceled)

24. A method of increasing the concentration of nitrate, or nitrite, in plasma in a subject with heart failure, the method comprising administering to the subject a therapeutically effective amount of a composition comprising at least one selected from the group consisting of inorganic nitrate, organic nitrate, inorganic nitrite, or organic nitrite.

25. The method of claim 24, wherein the heart failure is heart failure with preserved ejection fraction (HFpEF).

26. The method of claim 24, wherein the composition comprising at least one selected from the group consisting of inorganic nitrate, organic nitrate, inorganic nitrite, or organic nitrite is a liquid comprising at least a part of at least one nitrate-containing vegetable.

27. The method of claim 26, wherein the at least a part of at least one nitrate-containing vegetable is beetroot.

28-52. (canceled)
